(19) United States
(12) Patent Application Publication
(10) Pub. No.: US 2025/0090226 A1
(43) Pub. Date: Mar. 20, 2025

(54) ABLATION CATHETER WITH CUT ELECTRODE

(71) Applicant: Boston Scientific Scimed, Inc., Maple Grove, MN (US)

(72) Inventors: Joseph Walker, Shoreview, MN (US); Joel T. Eggert, Plymouth, MN (US)

(21) Appl. No.: 18/885,308

(22) Filed: Sep. 13, 2024

Related U.S. Application Data
(60) Provisional application No. 63/583,024, filed on Sep. 15, 2023.

(51) Int. Cl. A61B 18/14; A61B 17/00; A61B 18/00

(52) U.S. Cl. CPC A61B 18/1492; A61B 2017/00862; A61B 2017/00867; A61B 2018/00083; A61B 2018/00136; A61B 2018/00357; A61B 2018/00577; A61B 2018/00613; A61B 2018/1417; A61B 2018/142; A61B 2018/1467

(57) ABSTRACT

A catheter for ablating cardiac tissue through irreversible electroporation includes a tubular shaft and an electrode assembly extending distally from a tubular shaft. The electrode assembly defines a distally located flexible central hub portion and flexible splines. A distal ablation electrode includes a hub portion and ablation electrode radial segments. Each ablation electrode radial segment includes a first series of gaps and a second series of gaps. The first series of gaps are spaced from one another along a length of the ablation electrode radial segment and extend partially across the width of the ablation electrode radial segment from a first longitudinal side toward a second longitudinal side. The second series of gaps are spaced from one another along the length of the ablation electrode radial segment and extend partially across the width of the ablation electrode radial segments from the second longitudinal side toward the first longitudinal side.

### CROSS REFERENCE TO RELATED APPLICATIONS

The present application claims priority to U.S. Provisional Patent Application No. 63/583,024, filed Sep. 15, 2023, the entire disclosure of which is incorporated herein by reference.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a catheter for ablating cardiac tissue through irreversible electroporation, the catheter comprising a tubular outer shaft and an electrode assembly extendable distally from the distal end of the tubular shaft, the electrode assembly defining a distally located flexible central hub portion and a plurality of flexible splines each including a distal end portion extending proximally from the central hub portion, a proximal end portion attached to and constrained by the shaft, and an intermediate portion between the proximal end portion and the distal end portion. A distal ablation electrode including an ablation electrode hub portion is located on the flexible central hub portion and a plurality of ablation electrode radial segments, each of the plurality of radial segments extending longitudinally to a corresponding proximal end along at least a section of a corresponding intermediate portion of the flexible splines. Each of the ablation electrode radial segments includes a first longitudinal side, a second longitudinal side and a width extending between the first and second longitudinal side, each ablation electrode radial segment including: a first series of gaps spaced from one another along a length of the ablation electrode radial segment and extending partially across the width of the ablation electrode radial segment from the first longitudinal side toward the second longitudinal side; and a second series of gaps spaced from one another along the length of the ablation electrode radial segment and extending partially across the width of the ablation electrode radial segments from the second longitudinal side toward the first longitudinal side.

In Example 2, the catheter of Example 1, wherein the electrode assembly includes a flexible circuit having a flexible circuit hub and a plurality of flexible circuit branches integrally formed with and extending proximally from the flexible circuit hub.

In Example 3, the catheter of Example 2, wherein the electrode assembly includes a shape-memory support member coupled to the flexible circuit.

In Example 4, the catheter of Example 3, wherein the shape-memory support member faces inwardly and opposite the ablation electrode on the electrode assembly.

In Example 5, the catheter of any of Examples 3 and 4, wherein the support structure is formed from a nickel-titanium alloy.

In Example 6, the catheter of any of Examples 1-5, wherein the first and second series of gaps are formed by mechanical cutting.

In Example 7, the catheter of any of Examples 1-5, wherein the first and second series of gaps are formed by laser cutting.

In Example 8, the catheter of any of Examples 1-7, wherein the first and second series of gaps are filled with a coating.

In Example 9, the catheter of Example 8, wherein the coating comprises parylene.

In Example 10, the catheter of any of Examples 1-9, wherein the distal ablation electrode is formed from copper.

In Example 11, the catheter of any of Examples 1-10, wherein the flex circuit further includes a hub sensing electrode centrally located on the flex circuit hub.

In Example 12, the catheter of any of Examples 1-11, further comprising one or more shaft electrodes located proximate the distal end of the tubular outer shaft.

In Example 13, the catheter of Example 12, wherein the distal ablation electrode and the one or more shaft electrodes are configured to define an anode/cathode electrode pair for delivery of electroporation ablation energy to target tissue.

In Example 14, the catheter of any of Examples 1-13, wherein the electrode assembly further comprises a support member having a support member hub and a plurality of support member branches extending proximally from the support member hub, wherein the flex circuit hub is disposed over the support member hub, and each of the flex circuit branches is disposed over a respective one of the support member branches.

In Example 15, the catheter of Example 14, wherein the electrode assembly includes a first region including an adhesive layer disposed between and mechanically attaching the flexible circuit to the support member, and a second region in which the flexible circuit and the support member are not directly mechanically attached together.

In Example 16, a catheter for ablating cardiac tissue through irreversible electroporation, the catheter comprising: a tubular shaft having a distal end; an electrode assembly extendable distally from the distal end of the tubular shaft, the electrode assembly defining a distally located flexible central hub portion and a plurality of flexible splines each including a distal end portion extending proximally from the central hub portion, a proximal end portion attached to and constrained by the shaft, and an intermediate portion between the proximal end portion and the distal end portion; a distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines; wherein each of the ablation electrode radial segments includes a first longitudinal side, a second longitudinal side and a width extending between the first and second longitudinal side, each ablation electrode radial segment including: a first series of gaps spaced from one another along a length of the ablation electrode radial segment and extending partially across the width of the ablation electrode radial segment from the first longitudinal side toward the second longitudinal side; and a second series of gaps spaced from one another along the length of the ablation electrode radial segment and extending partially across the width of the ablation electrode radial segments from the second longitudinal side toward the first longitudinal side.

In Example 17, the catheter of Example 16, wherein the electrode assembly includes a flexible circuit having a flexible circuit hub and a plurality of flexible circuit branches integrally formed with and extending proximally from the flexible circuit hub.

In Example 18, the catheter of Example 17, wherein the electrode assembly includes a shape-memory support member coupled to the flexible circuit.

In Example 19, the catheter of Example 18, wherein the shape-memory support member faces inwardly and opposite the ablation electrode on the electrode assembly.

In Example 20, the catheter of Example 18, wherein the support structure is formed from a nickel-titanium alloy.

In Example 21, the catheter of Example 16, wherein the first and second series of gaps are formed by mechanical cutting.

In Example 22, the catheter of Example 16, wherein the first and second series of gaps are formed by laser cutting.

In Example 23, the catheter of Example 16, wherein the first and second series of gaps are filled with a coating.

In Example 24, the catheter of Example 23, wherein the coating is parylene.

In Example 25, the catheter of Example 16, wherein the distal ablation electrode is formed from copper.

In Example 26, the catheter of Example 16, wherein the flex circuit further includes a hub sensing electrode centrally located on the flex circuit hub.

In Example 27, the catheter of Example 16, further comprising one or more shaft electrodes located proximate the distal end of the tubular outer shaft.

In Example 28, the catheter of Example 27, wherein the distal ablation electrode and the one or more shaft electrodes are configured to define an anode/cathode electrode pair for delivery of electroporation ablation energy to target tissue.

In Example 29, catheter of Example 16, wherein the electrode assembly further comprises a support member having a support member hub and a plurality of support member branches extending proximally from the support member hub, wherein the flex circuit hub is disposed over the support member hub, and each of the flex circuit branches is disposed over a respective one of the support member branches.

In Example 30, a catheter for ablating cardiac tissue through irreversible electroporation, the catheter comprising: a tubular outer shaft having a proximal end and an opposite distal end; an electrode assembly extending distally from the distal end of the outer shaft, the electrode assembly defining a distally-located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising: a support member formed from a superelastic material and having a support member hub and a plurality of support member branches integrally formed with and extending proximally from the support member hub; a flexible circuit disposed over an outer surface of the support member and having a flex circuit hub disposed over the support member hub, and a plurality of flex circuit branches integrally formed with the flex circuit hub, each of the flex circuit branches disposed over a respective one of the support member branches, the flexible circuit further including: an outwardly-facing distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines; wherein each of the ablation electrode radial segments includes a longitudinally extending serpentine conductive path from the ablation electrode hub portion to the proximal end through a plurality of laterally extending gaps.

In Example 31, the catheter of Example 30, wherein the laterally extending gaps are formed by mechanical cutting.

In Example 32, the catheter of Example 30, wherein the laterally extending gaps are filled with a coating.

In Example 33, the catheter of Example 30, wherein the distal ablation electrode is formed from copper.

In Example 34, catheter for ablating cardiac tissue through irreversible electroporation, the catheter comprising: a tubular outer shaft having a proximal end and an opposite distal end; and an electrode assembly extendable distally from the distal end of the tubular shaft, the electrode assembly defining a distally located flexible central hub portion and a plurality of flexible splines each including an intermediate portion, the electrode assembly comprising, an outwardly-facing distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines; wherein each of the ablation electrode radial segments includes a longitudinally extending conductive path from the ablation electrode hub portion to the proximal end through a plurality of laterally extending gaps extending only partially across the radial segments.

In Example 35, the catheter of Example 34, wherein the laterally extending gaps are filled with a coating.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2A is a perspective illustration of a distal portion of a splined catheter for use in the electrophysiology system of FIG. 1, in accordance with embodiments of the subject matter of the disclosure.

FIGS. 2B and 2C are partial plan views an electrode assembly of the splined catheter of FIG. 2A. shown in two-dimensions, in accordance with embodiments of the subject matter of the disclosure.

FIG. 2D is a partial plan view of a feature of an electrode assembly of the splined catheter of FIG. 2A.

FIGS. 3 and 4 are plan views of alternative electrode assemblies for use in the catheter of FIG. 1, in accordance with embodiments of the subject matter of the disclosure.

FIG. 5. is a schematic view of a distal portion of a splined catheter constructed in accordance with embodiments of the subject matter of the disclosure in a collapsed configuration.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

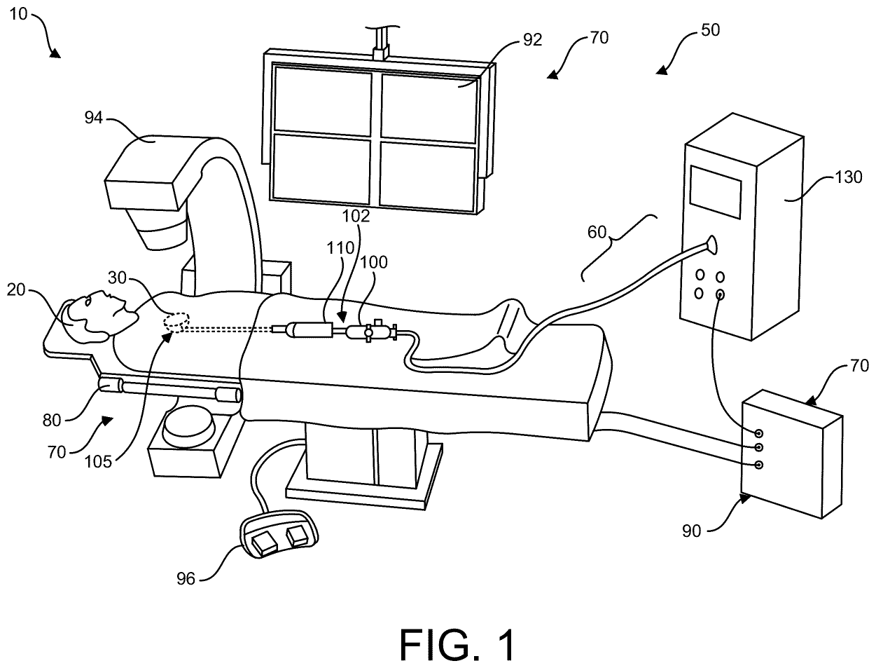

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

In embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control or perform the functional aspects of the electroporation catheter system 60. In embodiments, the memory can be part of the one or more controllers, microprocessors, or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

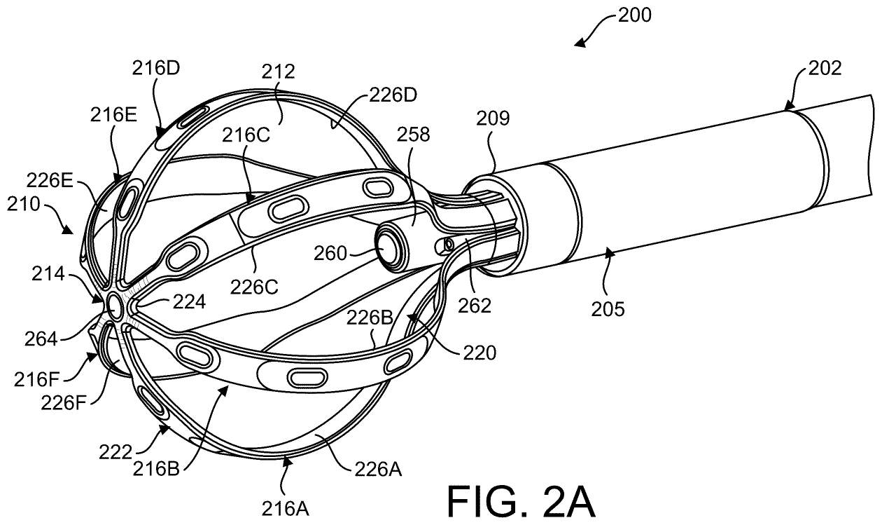

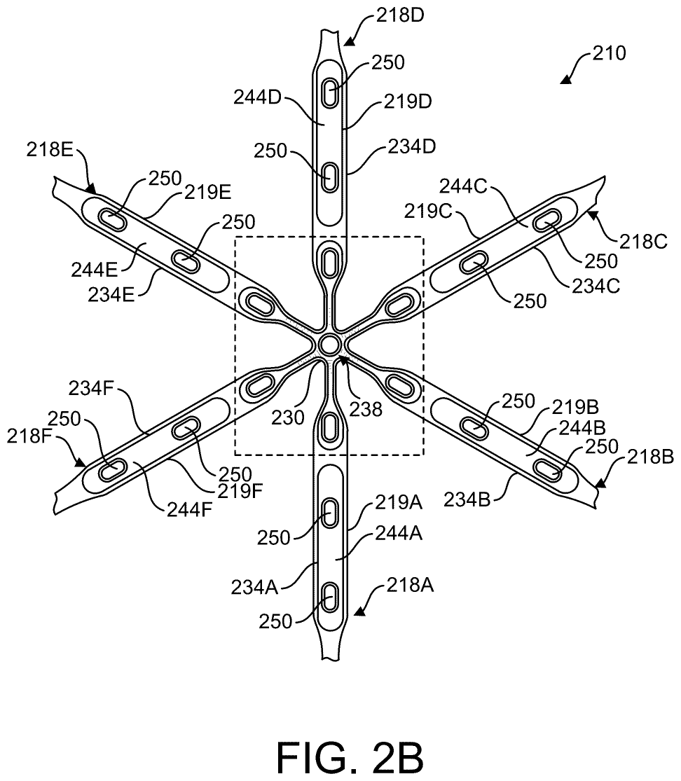

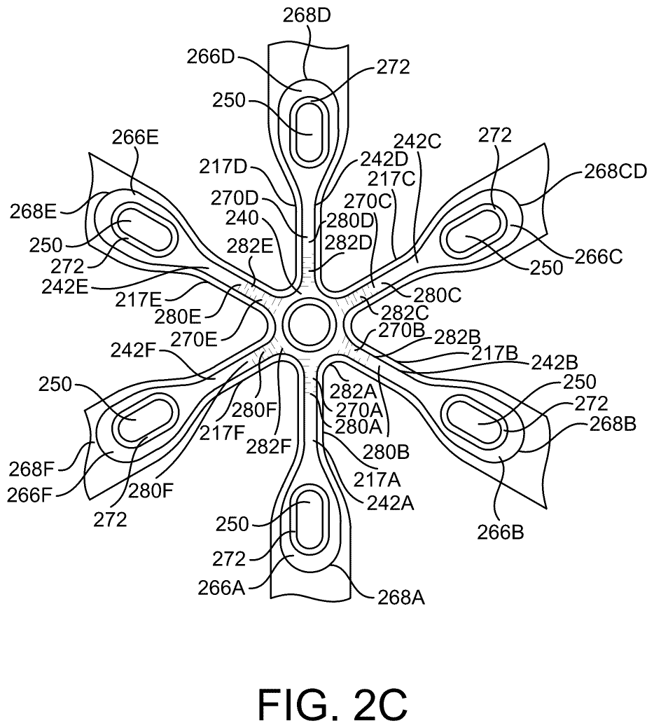

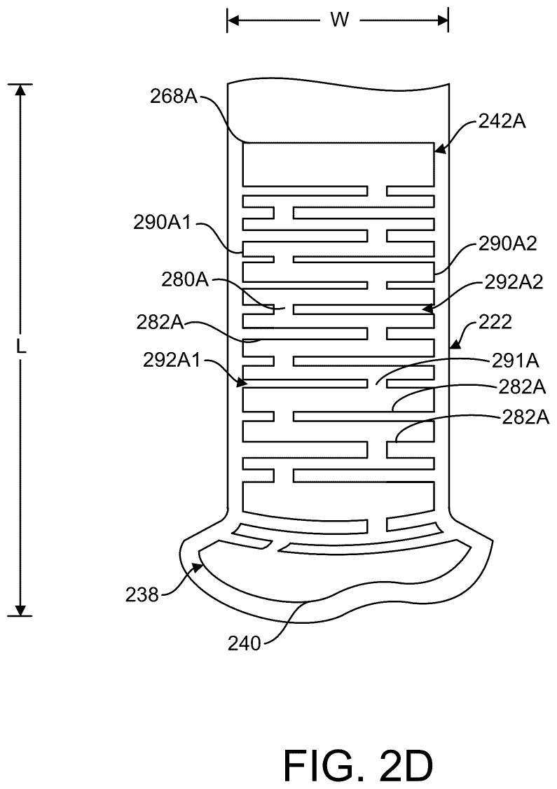

FIG. 2A partial perspective illustration of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 208 having a shaft distal end 209, and an electrode assembly 210 extending distally from the distal end 209 of the outer shaft 208. In embodiments, the electrode assembly 210 is configured to self-expand from a collapsed configuration when constrained within a delivery sheath to a pre-defined expanded configuration defining an inner space 212. In some embodiments, the electrode assembly includes multiple ablation electrodes configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

In embodiments, the electrode assembly 210 is primarily designed for the creation of relatively localized ablation lesions (i.e., focal lesions), as compared to relatively large diameter circumferential lesions created in pulmonary vein isolation procedures. The teachings of the present disclosure, however, can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

As discussed below, the electrode assembly 210 defines a distally located flexible central hub portion 214 and a plurality of flexible splines 216A-216F each including a distal end portion 217A-217F extending proximally from the central hub portion 214, a proximal end portion 218A-218F attached to and constrained by the shaft 208, and an intermediate portion 219A-219F between the proximal end portion 218A-218F and the distal end portion 217A-217F. The electrode assembly comprises an outwardly-facing distal ablation electrode 238 including an ablation electrode hub portion 240 located on the flexible central hub portion 214 and a plurality of ablation electrode radial segments 242A-242F, each of the plurality of radial segments 242A-242F extends longitudinally to a corresponding proximal end 268A-268F along at least a section of a corresponding intermediate portion 219A-219F of the flexible splines 216A-216F.

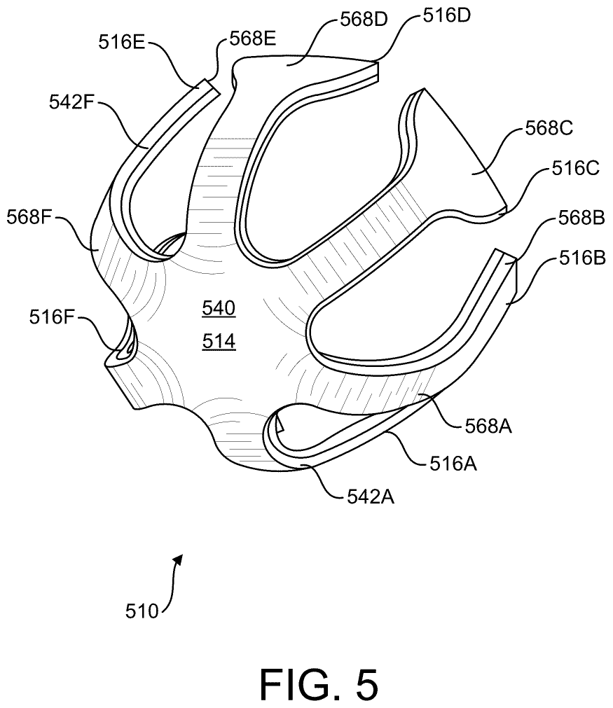

During operation, the portions of the splines 216A-216F proximate the central hub portion 214 are deflected and stressed within the delivery sheath (see, e.g., FIG. 5). The conductive materials used to construct the distal ablation electrode 240 may undergo plastic deformation in the collapsed configuration. In some situations, the device may be retracted into the sheath and released again. The distal ablation electrode 240 may undergo further plastic deformation during each such sheathing process. Over time, this process may effect the shape of the electrode assembly in the expanded configuration. To mitigate this effect and reduce the potential for plastic deformation, each of the ablation electrode radial segments 242A-242F includes series of laterally expending gaps. As further described below, such gaps may form a winding or serpentine configuration or may be formed of an alternating series of gaps extending laterally inward from opposing lateral edges of the radial segments.

FIGS. 2B-2C are partial plan views the electrode assembly 210 of the electroporation catheter 200, shown in two-dimensions to illustrate the layout of the electrode assembly 210. Referring to FIGS. 2A-2C together, in the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown in the illustrated embodiment, the intermediate portion 219A-219F of each spline 216A-216F has a lateral width that is greater than the lateral width of each of the respective proximal end portion 218A-218F and the distal end portions 217A-217F. In embodiments, the particular geometry of the splines 216A-216F and the related components, e.g., ablation and mapping electrodes, is optimized to provide desired mechanical and therapeutic/diagnostic capabilities. In the embodiments described and illustrated, each of the electrode assemblies has six splines. This is for illustration purposes, and for a given clinical application, more or fewer than six splines may be included.

In the illustrated embodiment, the splines 216A-216F are composed of a support member 220 and a flexible circuit 222 secured to and disposed over an outer surface of the support member 222. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy.

The support member 220 includes a support member hub 224 and a plurality of support member branches 226A-226F. In embodiments, the support member branches 226A-226F are integrally formed with and extend proximally from the support member hub 224. For example, the entire support member 200 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape such as shown in FIG. 2A due to the shape memory properties of the material, while providing sufficient flexibility necessary to collapse the electrode assembly 210 within a delivery sheath. In embodiments, the support member branches 226A-226F can be selectively configured along their lengths to tune the mechanical characteristics of the electrode assembly 210.

The flexible circuit 222 includes a flex circuit hub 230 and a plurality of flex circuit branches 234A-234F. In embodiments, the flex circuit hub 230 is disposed over and secured to the inwardly-facing support member hub 224. In embodiments, the flex circuit branches 234A-234F are integrally formed with the flex circuit hub 230, and each of the flex circuit branches 234A-234F is disposed over and secured to a respective one of the inwardly-facing support member branches 226A-226F. The flexible circuit 222 comprises a layered construction including one or more dielectric substrate layers, and conductive traces formed thereon. In some embodiments, the flex circuit branches 234A-234F comprise a layered structure that, is typical of flexible circuits for use in medical device electrode assemblies. For instance, the flex circuit branches 234A-234F include a dielectric base layer disposed over the support member branches 226A-226F, an optional inner flexible adhesive layer over the base layer, a conductive trace layer over the adhesive layer (when present), and a dielectric upper layer over the conductive trace layer. The dielectric materials chosen for the layers and can be any conventional materials suitable for use in flexible circuits for medical devices, e.g., polyamides. Similar to the support member 220, the unitary construction of the flexible circuit 222 enhances its structural properties, for example, by minimizing joints or other discontinuities at regions subject to relatively high stresses during use.

As shown, the flexible circuit 222 includes a distal ablation electrode 238 that has a distal ablation electrode hub portion 240 and a plurality of radial segments 242A-242F. In the illustrated embodiment, the distal ablation electrode hub portion 240 is located on the flex circuit hub 230. Additionally, the radial segments 242A-242F are integrally formed with the distal ablation electrode hub portion 240. Each of the radial segments 242A-242F extends proximally along a portion of a respective one of the flex circuit branches 234A-234F. The flexible circuit 222 further includes a plurality of proximal ablation electrodes 244A-244F. As shown, each of the proximal ablation electrodes 244A-244F is located on a respective one of the flex circuit branches 234A-234F. In the embodiments, the distal ablation electrode 238 is located opposite the flexible circuit 222 from the support member 220, and the distal ablation electrode 238 is outwardly-facing.

As further shown, the flexible circuit 222 includes a plurality of spline sensing electrodes 250. In the illustrated embodiment, each of the spline sensing electrodes 250 is disposed within a periphery of one of the proximal ablation electrodes 244A-244F or one of the radial segments 242A-242F of the distal ablation electrode 238. For example, as shown, each of the distal-most spline sensing electrode 250 is disposed within a periphery of a respective one of the radial segments 242A-242F of the distal ablation electrode 238 and is electrically isolated from the distal ablation electrode 238. Additionally includes a plurality of the more proximally-located spline sensing electrodes 250 is disposed along and within a periphery of a respective one of each of the proximal ablation electrodes 244A-244F and electrically isolated therefrom.

In some embodiments, the structural functionality of the support member 220 can be provided by a suitably designed flexible circuit 222. As such, although the electrode assembly 210 is described in detail as including the support member 220 as a primary structural member, in other embodiments the support member 220 can be omitted in its entirety and the corresponding functionality can be provided by the flexible circuit 222.

In the particular illustrated embodiment, the electroporation catheter 200 includes a central post 258 extending distally from the distal end 209 of the outer shaft 202. As shown, the central post 258 extends partially into the inner space 212 and includes a post electrode 260. As further shown in the illustrated embodiment, an optional irrigation lumen 261 is supported by the central post 258. In embodiments, the central post 258 may house additional components. For example, in embodiments, a magnetic navigation sensor (not shown) may be partially or wholly disposed within the central post 258. However, in other embodiments such a sensor may be located elsewhere on the electroporation catheter 200 (e.g., within the outer shaft 202). In the illustrated embodiment, the electrode assembly 210 further includes a hub sensing electrode 264 centrally located on the flex circuit hub 230.

The post electrode 260 can provide a number of functional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70 (FIG. 1).

In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250. In some embodiments, the hub sensing electrode 264 can be omitted in its entirety and replaced with a distal ablation electrode having no centrally located aperture on the flex circuit hub 238.

In the various embodiments, each of the respective proximal ablation electrodes 244A-244F, the distal ablation electrode 238, the spline sensing electrodes 250 and the hub sensing electrode 264 are separately electrically connected to the control system of the electroporation console 130 (FIG. 1) and are individually addressable to provide for a wide range of ablation and sensing modes, e.g., monopolar and bipolar modes. During monopolar ablation operation, an ablation electrode, a group of ablation electrodes, or collectively all of the ablation electrodes on the electrode assembly 210 are electrically coupled in common and configured to operate at one polarity, and an electrode located elsewhere (e.g., a dispersive electrode located on the patient, typically on the back, buttocks, or other suitable anatomical location, or an electrode on a different catheter or probe located outside the cardiac chamber in which the electrode assembly 210 is located) is configured to operate at the opposite polarity. In one example, the distal ablation electrode 238 and all of the proximal ablation electrodes 244A-244F are configured to be electrically in common as an anode or cathode, and an extracorporeal dispersive electrode located on a back patch is configured as the other of the cathode or anode. In other examples, selected proximal ablation electrodes, and optionally the distal ablation electrode 238, can be configured to operate together as an anode or cathode, and the extracorporeal dispersive electrode is configured as the other of the cathode or anode. The preceding example can advantageously allow for selective steering of the resulting electric fields to optimize electroporation effectiveness based on the relative orientation of the ablation assembly 210 and the target tissue. A wide range of monopolar ablation electrode configurations may be utilized.

During bipolar ablation operation, a first set of one or more ablation electrodes of the electrode assembly 210 is configured as the anode (or cathode) and a second set of one or more other ablation electrodes of the electrode assembly 210 is configured as the cathode (or anode). In examples, the bipolar ablation electrode sets can comprise electrodes on different splines or can be formed between one or more of the proximal ablation electrodes and the distal ablation electrode.

In a similar manner, any of the spline sensing electrodes 250, the post reference electrode 260 or the hub sensing electrode 264 can also be individually addressed for bipolar sensing and mapping an any number of combinations. Additionally, in embodiments, individual addressability allows any of the spline sensing electrodes and/or the hub sensing electrode 264 to be configured by the control system as ablation electrodes to operate in conjunction with the distal ablation electrode 238 and any of the proximal ablation electrodes 244A-244F, in either monopolar or bipolar mode.

Referring in particular to FIG. 2C, each of the radial segments 242A-242F of the distal ablation electrode 238 includes proximal portion 266A-266F having a proximal end 268A-268F, and a distal portion 270A-270F extending longitudinally from the distal ablation electrode hub portion 240. As further shown, a radial segment aperture 272 is formed in each proximal portion 266A-266F, and a respective one of the distal-most spline sensing electrodes 250 is disposed within each of the radial segment apertures 272. In the illustrated embodiment, each of the proximal portions 266A-266F has a greater lateral width than that of the corresponding distal portion 270A-270F, at least in part to accommodate the distal-most spline sensing electrodes 250. In one example, central hub portion 214 includes the structures and thicknesses of the support member 220 with a thickness of approximately 76 micrometers, the flex circuit 222 with a thickness also of approximately 76 micrometers, and the distal ablation electrode 240 with a thickness of approximately eight micrometers.

In the collapsed configuration, the splines 216A-216F are flattened relative to their bulbous or generally spherical shape in the expanded configuration, and electrode assembly is configured to fit within a delivery sheath. The portions of the splines 216A-216F proximate the central hub portion 214 are abruptly deflected and stressed within the delivery sheath. For example, the central hub portion 214 may extend near orthogonally to the splines 216A-216F and provide the greatest amount of deformation stress on the splines 216A-216F just proximal to the central hub portion 214. While the support member 220 and flex circuit are constructed from materials configured to adapt to the stress and recover from the deformation as the electrode assembly 210 transitions between the collapsed configuration and the expanded configuration, the conductive materials used to construct the distal ablation electrode 240 are generally not as flexible and respond with a plastic deformation in the collapsed configuration. After only a handful of transitions between the collapsed configuration and the expanded configuration, the electrode assembly 210 can undergo a significant flattening to its shape in the expanded configuration from the intended bulbous profile.

Also referring in particular to FIG. 2C, each of the radial segments 242A-242F of the distal ablation electrode 238 includes a longitudinally extending serpentine conductive path 280A-280F from the distal ablation electrode hub portion 240 to the proximal end 268A-268F formed through a plurality of spaced-apart, laterally-extending gaps 282A-282F on the radial segments 242A-242F, in which each of the laterally-extending gaps 282A-282F extend only partially across the radial segments 242A-242F. For example, a laterally-extending gap of the plurality of laterally-extending gaps 282A-282F on a radial segment can be created from one side of the radial segment 242A-242F, the other side, or both and leaving a conductive piece in between such that no laterally-extending gap extends all of the way across the radial segment 242A-242F of the distal ablation electrode 238. In some embodiments, the gaps can be formed by a mechanical cutting process cutting laterally-extending gaps in the formed radial segments, a laser cutting process that may remove more material from the formed radial segment in each of the laterally-extending gaps, or by forming or casting the distal ablation electrode from the conductive material to include the gaps in the first instance. In some examples, the gaps 282A-282F are filled such as with a parylene coating. In some embodiments, the gaps 282A-282F extend completely though the depth of the distal ablation electrode 238 but do not extend into the flex circuit 222 beneath distal ablation electrode 238. In embodiments, the gaps 282A-282F are formed in high-stress regions of the distal ablation electrode 238 when subjected to the collapsed configurations, and the high stressed regions can extend beyond the radial segments 242A-242F to the distal ablation hub portion 240, and thus gaps 282A-282F may be included in or extended to the distal ablation hub portion 240 as well.

FIG. 2D illustrates a portion of the distal ablation electrode 238 including a radial segment such as radial segment 242A in the region between proximal end 268A and distal ablation hub portion 240. In the illustrated embodiment, the distal ablation electrode 238 is formed from a conductive pad, such as a copper pad, that is disposed on and electrically coupled to the flex circuit 222. The distal ablation electrode 238 includes an uninterrupted, longitudinally-extending conductive path 280A through the radial segment 242A from the distal ablation electrode 238 to the proximal end 268A. Each of the radial segments 242A-242F, as illustrated with respect to radial segment 242A, includes a first longitudinal side 290A1 and a second longitudinal side 290A2 and a plurality of laterally-extending gaps 282A spaced-apart along the longitudinal sides 290A to form the conductive path 280A. In the illustrated embodiment, more than one gaps can be formed at a latitude, such as a gap from the first longitudinal side 290A1 at the same latitude as a gap from the second longitudinal side 290A2 so as to leave a portion 291A of the conductive path 280A between the gaps (or between a gap and a longitudinal side). While the connecting portion 291A in FIG. 2D is shown inset from the lateral edge of the radial segment, in other embodiments, the connection portion 291A is disposed adjacent the longitudinal side.

In one embodiment, each ablation electrode radial segment, such as radial segment 242A, of the ablation electrode radial segments 242A-242F, includes a first longitudinal side 290A1, a second longitudinal side 290A2, and a width (W) extending between the first longitudinal side 290A1 and the second longitudinal side 290A2. Each ablation electrode radial segment such as radial segment 242A, includes a first series of gaps 292A1 and a second series of gaps 292A2. The first series of gaps 292A1 includes those gaps 282A that are spaced apart from one another along a length (L) of the ablation electrode radial segment 242A and extend partially across the width W of the ablation electrode radial segment 242A from the first longitudinal side 290A1 to the second longitudinal side 290A2. The second series of gaps 292A2 includes those gaps 282A that are spaced apart from one another along a length (L) of the ablation electrode radial segment 242A and extend partially across the width W of the ablation electrode radial segment 242A from the second longitudinal side 290A1 to the first longitudinal side 290A1. In one embodiment, gaps in the first series of gaps 292A1 and gaps in the second series of gaps 292A2 can share a latitude. In another embodiment, gaps in the first series of gaps 292A1 are interposed between gaps in the second series of gaps 292A2. In each embodiment, a serpentine conductive path 280A is formed between the gaps 282A along the length L of the ablation electrode radial segment 242A.

The characteristics of the laterally-extending gaps 282A-282F may be varied to adjust and impact the overall effect of the plastic deformation of the radial segments 242A-242F of the electrodes on the shape of the electrode assembly. According to various embodiments, the laterally-extending gaps 282A-282F may be disposed along substantially the entire distal portion 270 of the radial segments 242A-242F. In other embodiments, the gaps 282A-282F extend along substantially the entire length of the radial segments 242A-242F. In certain embodiments, the gaps 282A-282F are configured to remove between about 30 to 60 percent of the copper in the corresponding region of the radial segments of the electrodes. In various embodiments the laterally-extending gaps 282A-282F have a width of between about 20 to 50 percent of the width of the electrode material between the gaps. In certain embodiments, the gaps 282A-282F extend laterally from about 50 to 90 percent of the distance across the radial segment.

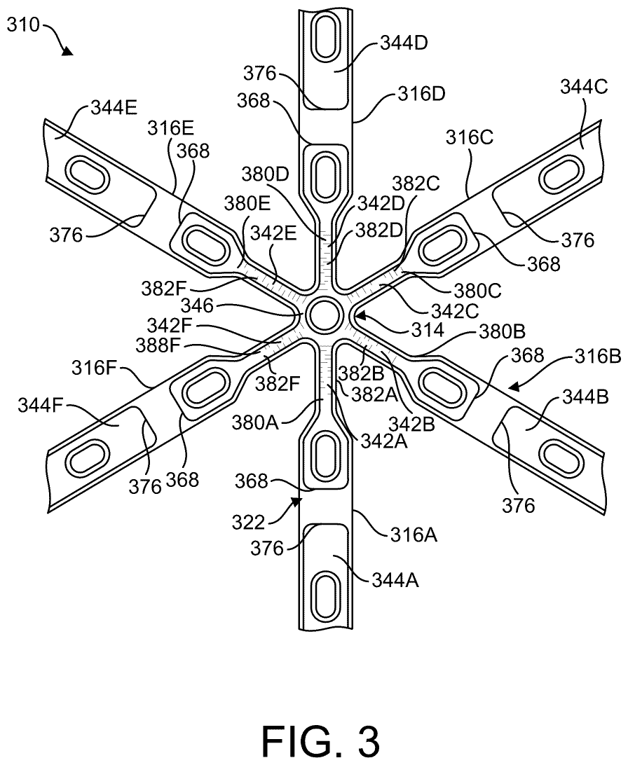

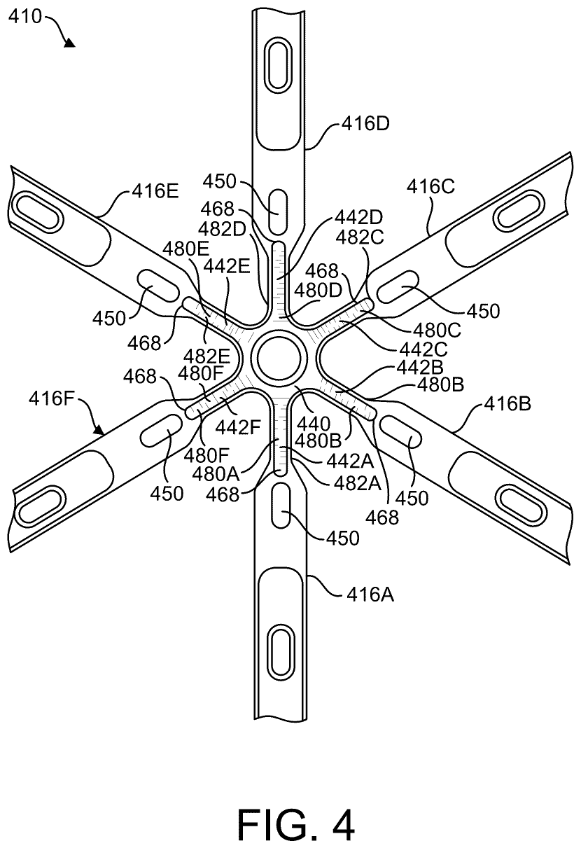

FIGS. 3-4 are illustrations of layouts of alternative electrode assemblies for use in the catheter of FIG. 1, in accordance with embodiments of the subject matter of the disclosure, such as alternate distal ablation electrodes. FIG. 3 illustrates a portion of an electrode assembly 310 that is substantially similar to the electrode assembly 210, and includes a central hub portion 314, a plurality of splines 316A-316F and a flex circuit 322. The embodiment of FIG. 3 differs from the electrode assembly 210 in that the proximal ends 368 of each distal ablation electrode radial segment 342A-342F is generally linear (i.e., is not semi-circular), and the distal end 376 of each of the proximal ablation electrodes 344A-344F is also generally linear, such that the opposing surfaces of the proximal end 368 of each radial segment 342A-342F and the opposing distal end of the corresponding proximal ablation electrode 344A-344F are generally parallel to one another. each of the radial segments 242A-242F of the distal ablation electrode 238 includes a longitudinally extending serpentine conductive path 380A-380F from the distal ablation electrode hub portion 340 to the proximal end 368A-368F formed amongst a plurality of spaced-apart, laterally-extending gaps 382A-382F on the radial segments 342A-342F, in which each of the laterally-extending gaps 382A-382F extend only partially across the radial segments 342A-342F.

FIG. 4 illustrates a portion of an electrode assembly 410 that includes a central hub portion 414, a plurality of splines 416A-416F and a flex circuit 422. As will be appreciated, the electrode assembly 410 also includes a support member (not shown) having substantially the same configuration of the support member 220 of the electrode assembly 210. The embodiment of FIG. 4 differs from the electrode assembly 210 in that the proximal end 468 of each distal ablation electrode radial segment 442A-442F terminates distally of and is spaced from the distal-most spline sensing electrode 450 on each spline. Each of the radial segments 442A-442F of the distal ablation electrode 438 includes a longitudinally extending serpentine conductive path 480A-480F from the distal ablation electrode hub portion 440 to the proximal end 468A-468F formed amongst a plurality of spaced-apart, laterally-extending gaps 482A-482F on the radial segments 442A-442F, in which each of the laterally-extending gaps 482A-482F extend only partially across the radial segments 442A-442F.

FIG. 5 illustrates a portion of an electrode assembly 510 in a collapsed configuration. The electrode assembly 510 includes a central hub portion 514, a plurality of spline 516A-516F, and a flex circuit 522. As will be appreciated, the electrode assembly 510 also includes a support member (not shown) having substantially the same configuration of the support member 220 of the electrode assembly 210. The portions of the splines 516A-516F proximate the central hub portion 514 are abruptly deflected and stressed within the delivery sheath. For example, the central hub portion 514 may extend near orthogonally to the splines 516A-516F and provide the greatest amount of deformation stress on the splines 516A-516F just proximal to the central hub portion 514. The proximal end 568 of each distal ablation electrode radial segment 542A-542F terminates distally of and is spaced from the distal-most spline sensing electrode on each spline. Each of the radial segments 542A-542F of the distal ablation electrode 538 includes a longitudinally extending serpentine conductive path 580A-580F from the distal ablation electrode hub portion 540 to the proximal end 568A-568F formed amongst a plurality of spaced-apart, laterally-extending gaps 582A-582F on the radial segments 542A-542F, in which each of the laterally-extending gaps 582A-582F extend only partially across the radial segments 542A-542F.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating cardiac tissue through irreversible electroporation, the catheter comprising:
a tubular shaft having a distal end;
an electrode assembly extendable distally from the distal end of the tubular shaft, the electrode assembly defining a distally located flexible central hub portion and a plurality of flexible splines each including a distal end portion extending proximally from the central hub portion, a proximal end portion attached to and constrained by the shaft, and an intermediate portion between the proximal end portion and the distal end portion;
a distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines;
wherein each of the ablation electrode radial segments includes a first longitudinal side, a second longitudinal side and a width extending between the first and second longitudinal side, each ablation electrode radial segment including:
a first series of gaps spaced from one another along a length of the ablation electrode radial segment and extending partially across the width of the ablation electrode radial segment from the first longitudinal side toward the second longitudinal side; and
a second series of gaps spaced from one another along the length of the ablation electrode radial segment and extending partially across the width of the ablation electrode radial segments from the second longitudinal side toward the first longitudinal side.

2. The catheter of claim 1, wherein the electrode assembly includes a flexible circuit having a flexible circuit hub and a plurality of flexible circuit branches integrally formed with and extending proximally from the flexible circuit hub.

3. The catheter of claim 2, wherein the electrode assembly includes a shape-memory support member coupled to the flexible circuit.

4. The catheter of claim 3, wherein the shape-memory support member faces inwardly and opposite the ablation electrode on the electrode assembly.

5. The catheter of claim 3, wherein the support structure is formed from a nickel-titanium alloy.

6. The catheter of claim 1, wherein the first and second series of gaps are formed by mechanical cutting.

7. The catheter of claim 1, wherein the first and second series of gaps are formed by laser cutting.

8. The catheter of claim 1, wherein the first and second series of gaps are filled with a coating.

9. The catheter of claim 8, wherein the coating is parylene.

10. The catheter of claim 1, wherein the distal ablation electrode is formed from copper.

11. The catheter of claim 1, wherein the flex circuit further includes a hub sensing electrode centrally located on the flex circuit hub.

12. The catheter of claim 1, further comprising one or more shaft electrodes located proximate the distal end of the tubular outer shaft.

13. The catheter of claim 12, wherein the distal ablation electrode and the one or more shaft electrodes are configured to define an anode/cathode electrode pair for delivery of electroporation ablation energy to target tissue.

14. The catheter of claim 1, wherein the electrode assembly further comprises a support member having a support member hub and a plurality of support member branches extending proximally from the support member hub, wherein the flex circuit hub is disposed over the support member hub, and each of the flex circuit branches is disposed over a respective one of the support member branches.

15. A catheter for ablating cardiac tissue through irreversible electroporation, the catheter comprising:
a tubular outer shaft having a proximal end and an opposite distal end;
an electrode assembly extending distally from the distal end of the outer shaft, the electrode assembly defining a distally-located central hub portion and a plurality of splines each including a distal end portion extending proximally from the central hub portion, a proximal end portion attached to and constrained by the outer shaft, and an intermediate portion between the proximal end portion and the distal end portion, the electrode assembly comprising:
a support member formed from a superelastic material and having a support member hub and a plurality of support member branches integrally formed with and extending proximally from the support member hub;
a flexible circuit disposed over an outer surface of the support member and having a flex circuit hub disposed over the support member hub, and a plurality of flex circuit branches integrally formed with the flex circuit hub, each of the flex circuit branches disposed over a respective one of the support member branches, the flexible circuit further including:
an outwardly-facing distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines;
wherein each of the ablation electrode radial segments includes a longitudinally extending serpentine conductive path from the ablation electrode hub portion to the proximal end through a plurality of laterally extending gaps.

16. The catheter of claim 15, wherein the laterally extending gaps are formed by mechanical cutting.

17. The catheter of claim 15, wherein the laterally extending gaps are filled with a coating.

18. The catheter of claim 15, wherein the distal ablation electrode is formed from copper.

19. A catheter for ablating cardiac tissue through irreversible electroporation, the catheter comprising:
a tubular outer shaft having a proximal end and an opposite distal end; and
an electrode assembly extendable distally from the distal end of the tubular shaft, the electrode assembly defining a distally located flexible central hub portion and a plurality of flexible splines each including an intermediate portion, the electrode assembly comprising,
an outwardly-facing distal ablation electrode including an ablation electrode hub portion located on the flexible central hub portion and a plurality of ablation electrode radial segments, each of the plurality of radial segments extending longitudinally to a corresponding proximal end along at least a section of a corresponding intermediate portion of the flexible splines;
wherein each of the ablation electrode radial segments includes a longitudinally extending conductive path from the ablation electrode hub portion to the proximal end through a plurality of laterally extending gaps extending only partially across the radial segments.

20. The catheter of claim 19, wherein the laterally extending gaps are filled with a coating.
